(19) United States
(12) Patent Application Publication
(10) Pub. No.: US 2026/0237507 A1
(43) Pub. Date: Aug. 13, 2026

(54) INTELLIGENT FLUID MONITORING CUP SYSTEM FOR HEALTHCARE SETTINGS

(71) Applicant: Alyssa Sieber, Commerce City, CO (US)

(72) Inventor: Alyssa Sieber, Commerce City, CO (US)

(21) Appl. No.: 19/632,595

(22) Filed: Mar. 30, 2026

Related U.S. Application Data
(63) Continuation of application No. PCT/US2025/051755, filed on Oct. 21, 2025.
(60) Provisional application No. 63/713,488, filed on Oct. 29, 2024.

(51) Int. Cl. G16H 40/67

(52) U.S. Cl. CPC G16H 40/67

(57) ABSTRACT

An intelligent fluid monitoring cup system for healthcare settings comprises a cup body with at least 750mL capacity, double handles, and a twist-on lid providing rotational access for patients with limited mobility. The system incorporates a capacitive sensor capable of detecting fluid level changes less than 0.5mm, providing sub-millimeter precision measurement. A computing device with integrated screen displays real-time fluid intake data, NPO status indicators, and remaining allowances. The system features direct integration with hospital electronic medical records via HL7/FHIR interoperability standards, barcode patient-device association, and nurse verification workflows. Hospital-grade hygiene protocols include sterilizable shells or disposable liner systems for multi-patient clinical environments.

### CROSS REFERENCE TO RELATED APPLICATIONS

This application claims the benefit of and priority to U.S. Provisional Patent Application No. 63/713,488, filed Oct. 29, 2024, and Patent Cooperation Treaty Patent Application PCT/US25/51755, filed Oct. 21, 2025, each entitled "Intelligent Fluid Monitoring Cup System for Healthcare Settings," the entire contents of which are incorporated herein by reference.

### FILED OF THE INVENTION

The present invention relates generally to medical monitoring devices and healthcare information systems. More particularly, the invention relates to fluid intake monitoring systems for clinical healthcare settings that integrate with electronic medical records and provide real-time patient data management. The invention further relates to intelligent monitoring apparatus and methods for tracking and recording patient fluid consumption in hospital and clinical environments.

### BACKGROUND OF THE INVENTION

Fluid intake monitoring is a critical aspect of patient care, particularly for individuals with conditions such as congestive heart failure, kidney failure, or those preparing for surgery. Accurate tracking of fluid consumption is essential for several reasons. For congestive heart failure patients, excessive fluid intake can exacerbate their condition, potentially leading to fluid accumulation in the lungs and other parts of the body. Proper fluid management is crucial for these patients' recovery and well-being. Inaccurate fluid tracking can result in prolonged hospital stays, increasing both patient discomfort and healthcare costs. A typical mild exacerbation of congestive heart failure may require a 2-3 day hospitalization, costing tens of thousands of dollars per day in the United States.

Current fluid monitoring methods face several challenges that have persisted despite technological advances in healthcare. The search of existing solutions reveals that while consumer-grade "smart" hydration bottles have existed for years, they fail to address the unique requirements of the clinical environment, such as robust hygiene protocols, direct EMR integration, and patient-specific workflow features. Manual recording of fluid intake remains prone to errors and inconsistencies, with patients often needing to ask nurses about their remaining fluid allowance, which strains nursing resources.

Existing consumer smart cups, such as those disclosed in US 2017/0340147 A1 to Leech and US 9,382,107 B2 to Pacey, each of which are incorporated herein by reference, are fundamentally designed for personal wellness tracking rather than clinical applications. These devices lack essential features for hospital use, including: (1) ergonomic design elements such as double handles for weakened patients; (2) sterilizable shells or disposable liner systems for hospital hygiene protocols; (3) dedicated NPO (Nil Per Os) mode with clear on-screen indicators; (4) barcode systems for linking devices to specific patient EMRs; (5) direct communicative links using healthcare standards like HL7 or FHIR for integration with systems like Epic or Cerner; and (6) nurse verification workflows for data integrity.

Traditional cup designs present additional challenges for patient populations requiring fluid monitoring. Standard hospital cups are often too large and heavy for weakened patients to handle comfortably, and conventional lids that must be pulled up create accessibility barriers for patients with limited dexterity or strength. The updated twist-on lid design addresses these limitations by providing easier access through rotational motion rather than vertical lifting, which is particularly beneficial for patients with weakened grip strength or mobility issues.

Measurement accuracy represents another significant challenge in existing approaches. Early measurement systems relied on estimation-based methods or external measurement devices like measurement tape that provided limited precision and were subject to user error. The transition to sensor-based measurement systems addresses these limitations through automated, high-precision monitoring capabilities.

Traditional liquid sensing approaches face significant limitations that have not been adequately addressed in the prior art. Estimation-based measurement methods rely on visual assessment or timing mechanisms that are inherently imprecise and subject to substantial user error, particularly in clinical settings where accuracy is critical. External measurement devices, such as measurement tape systems, provide only limited precision and require manual intervention that introduces additional opportunities for error. Conventional liquid level detection systems suffer from susceptibility to environmental interference, including proximity effects from users or external objects, which can cause measurement drift and compromise accuracy. These approaches fail to achieve the sub-millimeter precision required for clinical applications where exact fluid volumes are essential for patient safety and treatment efficacy. The persistent reliance on manual estimation and measurement techniques demonstrates a fundamental gap in the art for automated, high-precision liquid sensing solutions suitable for healthcare environments.

The persistent gap between existing consumer devices and clinical requirements demonstrates the long-felt need for a comprehensive solution. Clinical hydration monitoring systems like those described in US 2009/0043222 A1 to Licandro, incorporated herein by reference, attempt to address hydration monitoring through bioelectrical impedance rather than direct fluid intake measurement, indicating that even clinical approaches have not successfully addressed the need for direct oral fluid intake monitoring in a hospital setting.

Healthcare IT integration represents another critical gap in existing solutions. However, no existing solution has successfully combined all necessary elements: precise measurement capabilities, clinical ergonomics and hygiene features, comprehensive clinical workflow software, and seamless integration with hospital electronic medical record systems.

These persistent challenges highlight the need for an innovative solution that can accurately monitor and record fluid intake, provide real-time information to patients and caregivers, incorporate advanced sensor technology for precise measurement, utilize improved container design for patient accessibility, and integrate seamlessly with existing hospital systems to ensure comprehensive clinical workflow management.

Accurate fluid intake monitoring represents a critical component of patient safety and clinical outcomes management, with direct financial implications for healthcare institutions. For congestive heart failure (CHF) patients, inaccurate fluid tracking leading to volume overload can trigger acute exacerbations requiring emergency interventions, with individual episodes costing tens of thousands of dollars for short-term hospitalizations. The cumulative financial burden of CHF-related readmissions exceeds $2.8 billion annually in the United States healthcare system.

Pre-operative NPO protocol violations represent another significant safety and financial risk, with inadvertent fluid consumption potentially requiring surgery postponement, operating room rescheduling, and extended pre-operative monitoring periods. Such incidents not only compromise patient safety through increased anesthesia risks but also generate substantial additional costs through resource utilization inefficiencies and schedule disruptions affecting multiple patients and clinical teams.

Consumer-grade smart hydration devices, while advancing personal wellness tracking, fundamentally fail to address the unique requirements of clinical healthcare environments. These devices typically utilize estimation-based measurement methods, such as inclinometer and timing mechanisms disclosed in US 9,382,107 B2 to Pacey, which lack the precision required for clinical fluid management where exact volumetric measurements are essential for medical decision-making.

The consumer device focus on smartphone application integration creates additional barriers to clinical implementation, as healthcare environments require direct integration with hospital information systems rather than personal mobile devices that may not be accessible to nursing staff or may violate patient privacy and hospital security protocols. The absence of clinical workflow features, including NPO mode indicators, nurse verification mechanisms, and hospital-grade hygiene protocols, renders consumer devices unsuitable for clinical implementation despite their technological sophistication in consumer applications.

### SUMMARY OF THE INVENTION

The present invention in accordance with an embodiment addresses the critical challenges in clinical fluid monitoring through an intelligent fluid monitoring cup system that combines advanced sensor technology, ergonomic design improvements, and seamless healthcare integration to provide accurate, real-time fluid intake tracking for patients in healthcare settings.

A primary advantage of the present invention in accordance with an embodiment lies in its implementation of capacitive sensor technology for precise fluid level measurement. The capacitive sensing approach provides liquid level sensing with resolution capabilities of less than 1mm, representing a substantial improvement over traditional estimation-based methods that are prone to user error and environmental interference. The capacitive sensor configuration utilizes changes in dielectric constant of the cup contents to accurately measure fluid levels, offering sensitivity capable of detecting changes in fluid level of less than 0.5mm, which provides approximately three times the sensitivity required for clinical applications. This high-precision measurement capability eliminates the inaccuracies associated with manual estimation and external measurement devices that have plagued existing fluid monitoring approaches.

The invention in accordance with an embodiment incorporates significant design improvements that address patient accessibility and usability challenges identified in traditional hospital fluid monitoring systems. A key advancement is the implementation of an improved twist-on lid design that replaces conventional lids requiring vertical lifting motion. This twist-on screw top configuration provides easier access through rotational motion rather than vertical lifting, which is particularly beneficial for patients with weakened grip strength, limited dexterity, or mobility issues commonly encountered in clinical settings. This design improvement directly addresses the accessibility barriers that prevent effective fluid monitoring in patient populations requiring such care.

The invention in accordance with an embodiment further advances clinical fluid monitoring through increased container capacity, with the cup body designed to hold at least 750mL compared to traditional monitoring cups with insufficient volume around 500mL. This larger capacity reduces refilling frequency while maintaining manageable weight for patient use, thereby minimizing opportunities for measurement error and workflow disruption that occur with frequent refilling cycles.

Integration advantages of the present invention in accordance with an embodiment include comprehensive compatibility with widely used hospital charting systems such as Epic, Cerner, and Meditech through direct communicative links using healthcare interoperability standards like HL7 and FHIR. The system incorporates a barcode identification system that enables scanning to link the device to specific patient electronic medical records, ensuring accurate data association and eliminating manual transcription errors. A nurse verification mechanism provides professional oversight to confirm automated readings before permanent recording in patient medical records, enhancing data integrity and patient safety.

The invention's software capabilities in accordance with an embodiment provide clinical workflow optimization through specialized features including a dedicated NPO (Nil Per Os) mode with clear visual indicators, configuration lock functions to prevent unauthorized changes, manual fluid intake recording for consumption outside the monitored cup, and patient discharge/reset functionality for efficient device preparation between patients. These features work synergistically to create a comprehensive clinical workflow management tool that addresses the unique requirements of hospital environments.

Hygiene and infection control advantages in accordance with an embodiment are achieved through the incorporation of either sterilizable shell components or disposable liner systems, allowing for efficient cleaning or replacement between patient uses while maintaining the integrity of electronic measurement components. The spill-proof design features, similar to those found in specialized drinking vessels, help prevent accidental spills and ensure accurate fluid intake monitoring, particularly beneficial for patients with weakened grip strength or limited mobility.

The ergonomic design improvements in accordance with an embodiment include double handles that provide enhanced stability and ease of use for patients with various physical limitations, addressing the challenges posed by standard hospital cups that are often too large and heavy for weakened patients to handle comfortably. The lightweight construction accommodates patients with reduced strength while maintaining sufficient capacity for clinical hydration monitoring requirements.

By providing these integrated advantages—precise capacitive sensing technology, improved accessibility through twist-on lid design, enhanced capacity, seamless healthcare IT integration, and comprehensive clinical workflow features—the present invention addresses the long-felt need for accurate, automated fluid intake monitoring in clinical settings. The invention has the potential to improve patient care, reduce healthcare provider burden, enhance data accuracy, and ultimately improve outcomes for patients with conditions requiring strict fluid management, such as congestive heart failure, kidney failure, and pre-surgical patients.

### BRIEF DESCRIPTION OF THE FIGURES

FIG. 1 illustrates a front view of the cup, showing the general shape and key control features including MENU, ON/OFF, and LOCK buttons of an embodiment of the invention.

FIG. 2a depicts a front assembled view highlighting the main display screen of the cup in normal operation mode, presenting essential information such as the patient's name ("Hello Alyssa"), hospital welcome message ("Welcome to Heart Hospital"), current time (4:30 PM), fluid intake limit (2000mL), remaining fluid allowance (550mL left), and time until limit reset (7:30 hours until reset) in accordance with an embodiment.

FIG. 2b shows a front view of the cup the display screen in NPO (Nil Per Os) mode, displaying the patient greeting and hospital information while prominently indicating "NRO" status when the patient is not allowed to consume anything by mouth in accordance with an embodiment.

FIG. 2c presents a front view of the cup displaying the menu interface of the cup, displaying various configuration options including "Change time," "Change limit," "Change current intake amount," "Change reset time," "Set to NPO mode," "Pair to charting system," and "History (for manual charting)" in accordance with an embodiment.

FIG. 3 presents a schematic diagram showing the data flow from the flow meter through measured data to the computing device on screen, and then wirelessly transferred and converted to HL7 format for export to the external electronic medical records system.

FIG. 4 shows a back view of the cup illustrating the positioning of the capacitive sensor positioned for fluid level detection in accordance with an embodiment.

FIG. 5 illustrates an intelligent fluid monitoring cup system configured with an optional disposable liner system that enables complete contamination elimination between patient uses while maintaining the integrated computing device, display screen, and clinical control interface in accordance with an embodiment of the invention

### DETAILED DESCRIPTION

An embodiment of the invention provides a comprehensive fluid monitoring system designed to address the challenges associated with tracking and managing fluid intake for patients in healthcare settings. The preferred embodiment of the invention comprises a specially designed cup with integrated electronic components and software features that work together to accurately monitor, record, and communicate fluid consumption data.

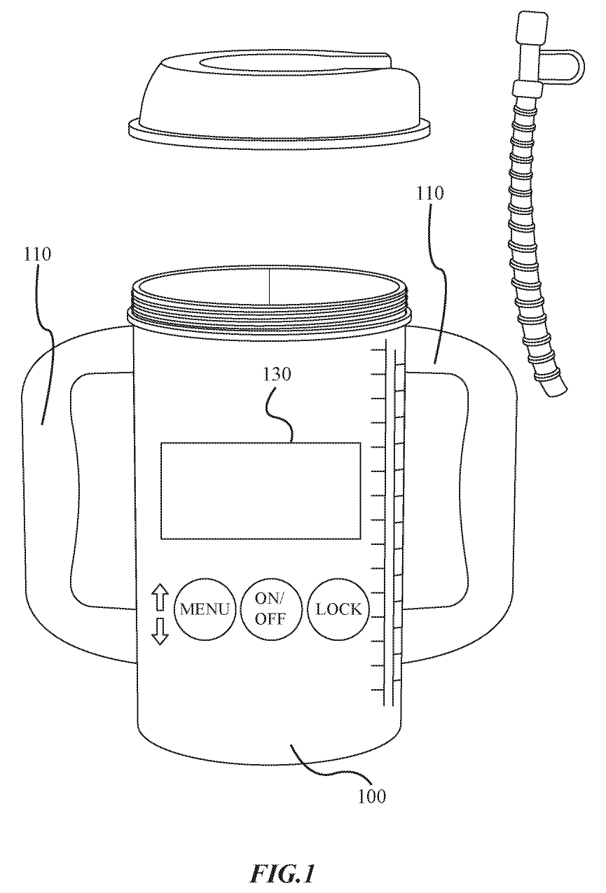

FIG. 1 illustrates a front view of the intelligent fluid monitoring cup system in accordance with an embodiment of the invention. The cup body (100) comprises a lightweight, ergonomic design with double handles (110) positioned on opposite sides to facilitate secure gripping by patients with weakened grip strength or limited mobility. The front face of the cup body (100) incorporates three primary control buttons positioned for easy access: a MENU button for accessing configuration options, an ON/OFF button for powering the system, and a LOCK button for preventing unauthorized changes to critical settings. This control interface design in accordance with an embodiment of the invention provides healthcare providers with intuitive access to system functions while maintaining security through the lock mechanism to prevent patient tampering with prescribed fluid monitoring parameters.

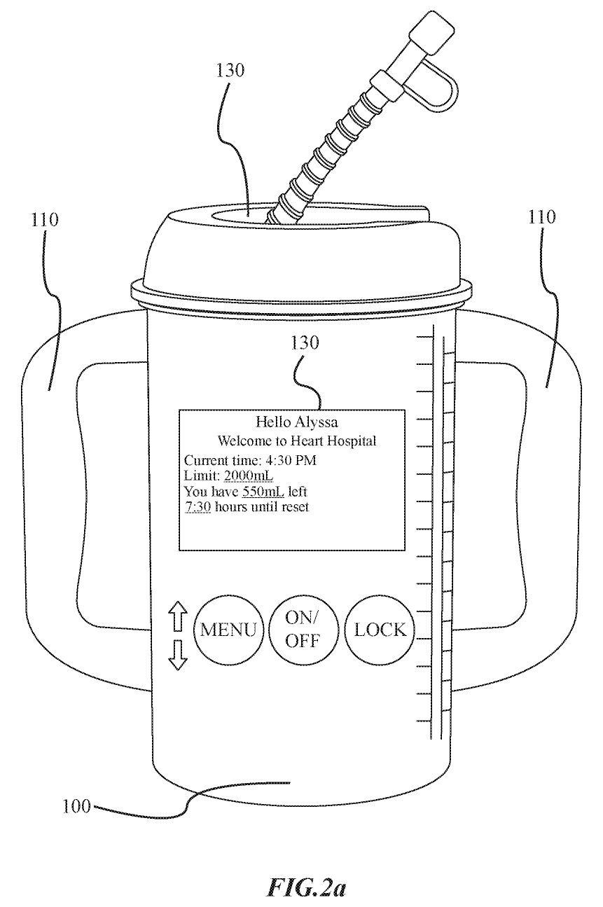

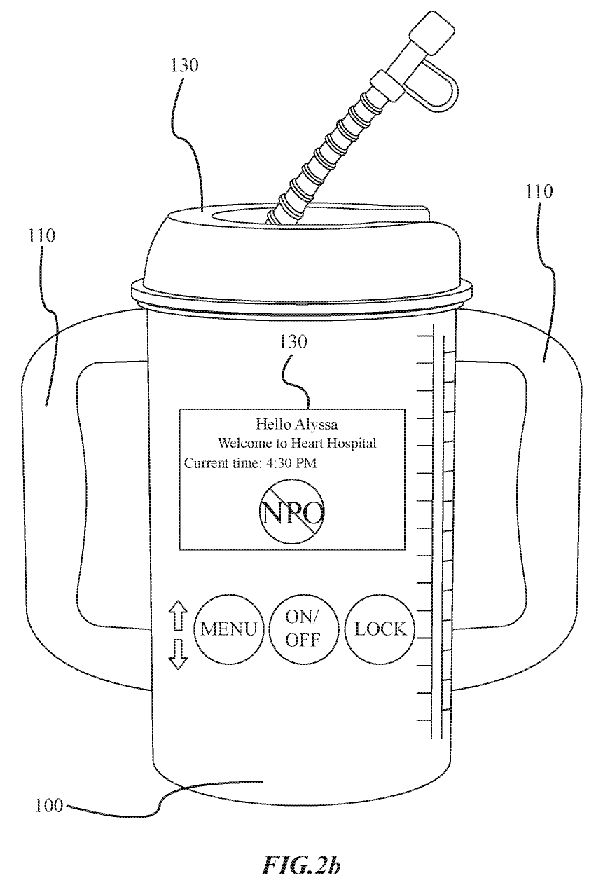

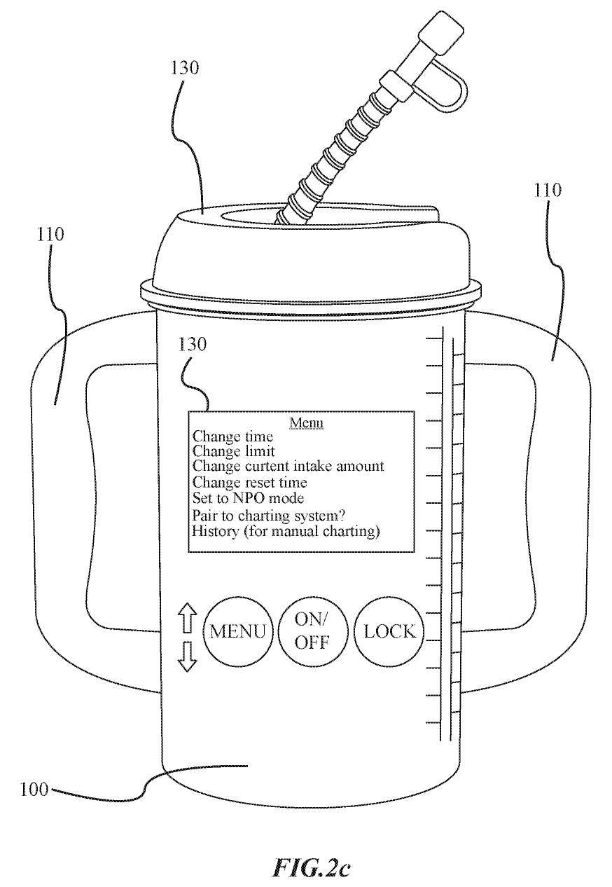

FIG. 2a depicts the main display screen (130) of the cup system in normal operation mode in accordance with an embodiment of the invention. The integrated screen (130) is positioned on the front face of the cup body (100) between the double handles (110) and presents essential clinical information in a clear, easily readable format. The display shows a personalized patient greeting ("Hello Alyssa"), hospital identification ("Welcome to Heart Hospital"), current time display (4:30 PM), the prescribed daily fluid intake limit (2000mL), remaining fluid allowance within the prescribed limit (550mL left), and countdown timer until the daily limit resets (7:30 hours until reset). This comprehensive information display in accordance with an embodiment of the invention enables patients, healthcare providers, and family members to immediately understand the patient's current fluid intake status and remaining allowance, promoting adherence to prescribed fluid restrictions and supporting effective clinical care management.

FIG. 2b shows the display screen (130) configured in NPO (Nil Per Os) mode in accordance with an embodiment of the invention. When activated by healthcare personnel, this specialized display mode presents the patient greeting and hospital identification information while prominently displaying "NRO" status as the central visual indicator. The NPO mode display in accordance with an embodiment of the invention provides clear, unambiguous communication that the patient is restricted from consuming any fluids by mouth, which is critical for pre-operative patients, those with specific medical conditions requiring fluid restriction, or patients with swallowing difficulties. The prominent visual indicator serves as an immediate reference for patients, family members, and healthcare staff, helping to prevent accidental fluid consumption that could compromise patient safety or treatment protocols.

FIG. 2c presents the menu interface displayed on the screen (130) when the MENU button is activated in accordance with an embodiment of the invention. The menu system provides healthcare personnel with access to various configuration options including "Change time" for adjusting the system clock, "Change limit" for modifying daily fluid intake restrictions, "Change current intake amount" for manual adjustment of recorded consumption, "Change reset time" for setting when daily limits restart, "Set to NPO mode" for activating fluid restriction status, "Pair to charting system" for establishing connectivity with hospital electronic medical records, and "History (for manual charting)" for reviewing past fluid intake data. This comprehensive menu system in accordance with an embodiment of the invention enables healthcare providers to customize the device settings according to individual patient needs and hospital protocols while maintaining the flexibility to manually record fluid consumption from sources outside the monitored cup.

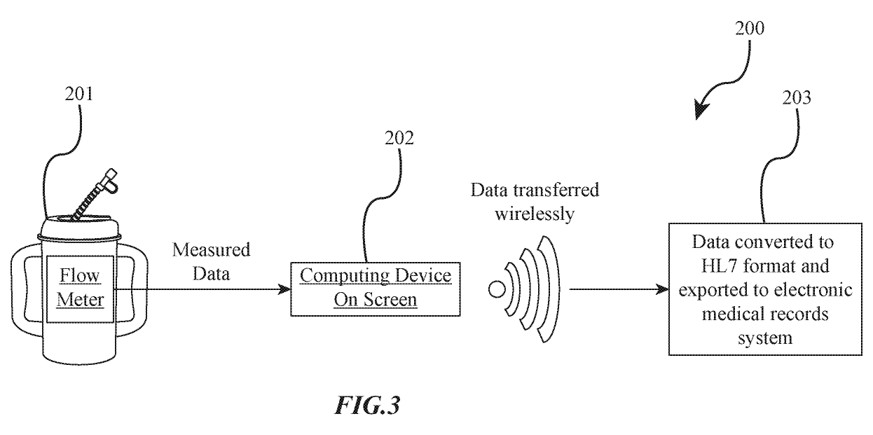

FIG. 3 illustrates a schematic data flow diagram showing the systematic processing and transmission of fluid measurement data in accordance with an embodiment of the invention. The flow meter (201) continuously monitors fluid consumption and generates measured data (202) representing the volume of liquid consumed by the patient. This measured data (202) is processed by the computing device on screen (203), which converts the raw sensor measurements into clinically meaningful fluid intake information and updates the display in real-time. The processed data is then wirelessly transferred and converted to HL7 format for seamless export to the external electronic medical records system. This automated data flow process in accordance with an embodiment of the invention eliminates manual data entry requirements, reduces transcription errors, and ensures that patient fluid intake information is immediately available to the healthcare team through existing hospital information systems, thereby enhancing clinical workflow efficiency and data accuracy.

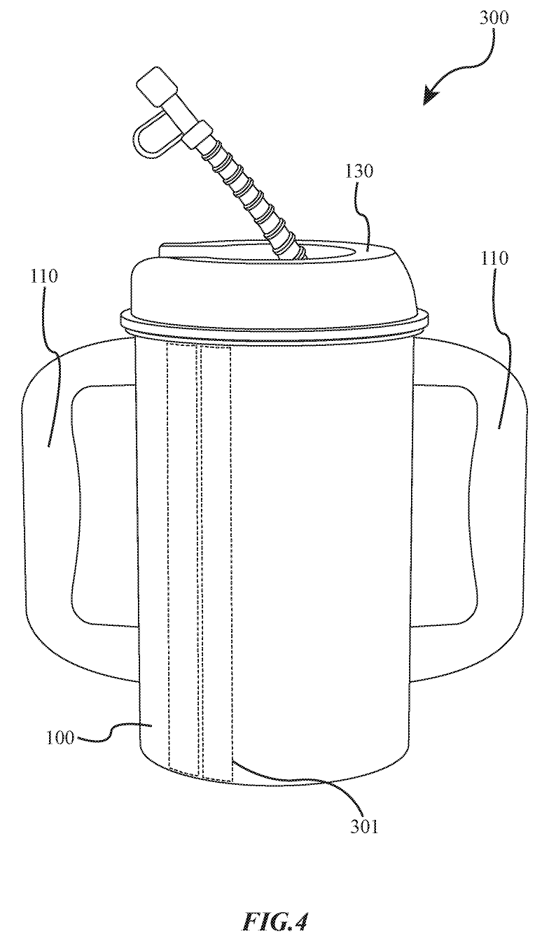

FIG. 4 shows the back view of the cup (300) illustrating the positioning of the capacitive sensor (301) for precise fluid level detection in accordance with an embodiment of the invention. The capacitive sensor (301) is strategically positioned on the exterior surface of the cup body (300) to provide optimal sensing capability for measuring fluid levels with sub-millimeter precision. The sensor (301) utilizes changes in dielectric constant of the cup contents to accurately determine fluid levels, offering sensitivity capable of detecting changes in fluid level of less than 0.5mm, which provides approximately three times the sensitivity required for clinical applications. The capacitive sensor implementation in accordance with an embodiment of the invention comprises a flexible sensor design that can be applied to the exterior of the cup with self-adhesive backing, connecting via a connector to the circuit board when the cup is assembled. This sensor configuration in accordance with an embodiment of the invention eliminates the susceptibility to environmental interference and proximity effects that compromise the accuracy of conventional liquid level detection systems, ensuring reliable and precise measurement of patient fluid consumption for critical clinical monitoring applications.

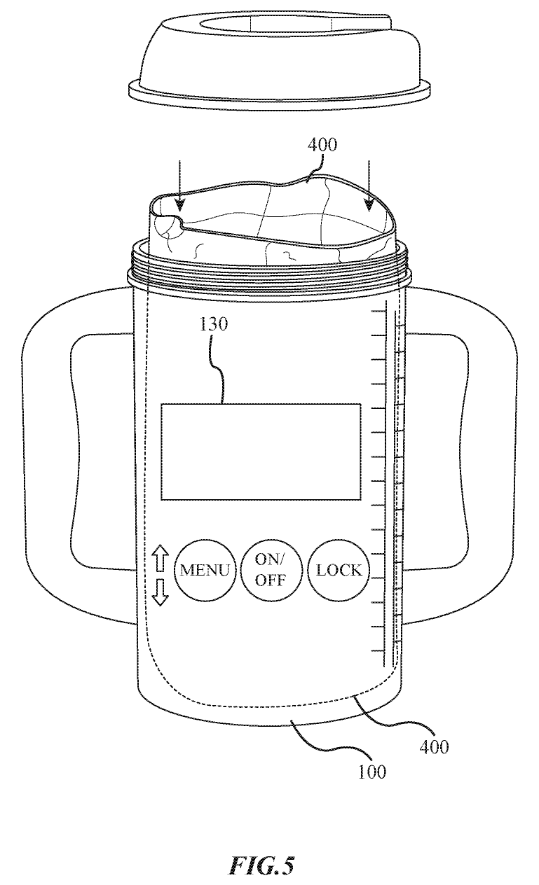

FIG. 5 illustrates a comprehensive system architecture and healthcare integration diagram in accordance with an embodiment of the invention, demonstrating the clinical workflow and enterprise-grade healthcare IT integration capabilities that distinguish the inventive solution from consumer wellness devices. The central fluid monitoring cup system (100) comprises the cup body with double handles and twist-on lid design, providing the foundational structure for the intelligent fluid monitoring apparatus. The integrated screen (130) positioned prominently on the cup system (100) serves as the primary patient interface, displaying clinical information including fluid allowance data, temporal information, patient safety indicators, and NPO status when activated, ensuring clear visual communication of critical fluid management data to patients, healthcare providers, and visitors. The optional liner (400) represents the disposable component system that enables complete contamination elimination between patient uses while preserving electronic measurement components, addressing the stringent hygiene requirements of multi-patient clinical environments through replaceable inner containers and disposable straw assemblies. The menu interface accessible through the "MENU" control enables healthcare personnel to configure patient-specific fluid restriction parameters, activate NPO mode, manually record additional fluid intake, and access comprehensive device settings through nurse-authenticated access protocols. The "ON/OFF" control provides power management functionality for the computing device and integrated measurement systems, enabling efficient battery conservation during periods of non-use while maintaining continuous monitoring capabilities when activated. The "LOCK" control implements the configuration lock mechanism that prevents unauthorized parameter modifications through hierarchical access control, ensuring that fluid restriction parameters established by physician orders cannot be altered by patients or visitors without appropriate clinical authorization. The barcode scanning workflow demonstrates the secure patient-device association process, utilizing GS1-compliant encoding standards with Code 128 or Data Matrix symbology to encode unique device identifiers that establish direct linkage with patient electronic medical records through hospital authentication protocols. The hospital EMR systems integration encompasses major healthcare information platforms including Epic, Cerner, and Meditech systems, each configured to receive and process fluid intake data through standardized healthcare interoperability protocols including HL7 v2.x and FHIR standards that format fluid intake observations as standardized message structures compatible with existing clinical documentation workflows. The nurse verification station provides authenticated healthcare personnel interface requiring clinical authorization before permanent recording of fluid intake data in patient legal medical records, implementing dual-factor authentication protocols and maintaining comprehensive audit trail functionality for regulatory compliance and quality assurance purposes. The NPO monitoring alerts system demonstrates critical patient safety functionality, providing automated notifications to nursing stations when patients with NPO status attempt to access fluids, displaying prominent visual warning indicators on the integrated screen (130) while simultaneously transmitting automated alerts to nursing station monitoring systems through the healthcare IT infrastructure. The comprehensive data flow architecture shows the sequential processing pathway from the capacitive sensor through the computing device to real-time display presentation and subsequent healthcare system integration, with data flow pathways demonstrating how measured fluid data is processed using algorithms that compensate for cup orientation, temperature variations, and fluid type differences before transmission to patient electronic medical records via healthcare-compliant data encryption protocols. The clinical workflow steps represent the complete patient care integration process beginning with barcode scanning for patient-device association, followed by patient record linkage within hospital EMR systems, continuous real-time monitoring through the capacitive sensor system, nurse verification protocols for data integrity confirmation, and permanent EMR recording of verified fluid intake information, transforming fluid monitoring from a manual, error- prone process into an automated, accurate, and efficient clinical tool that integrates seamlessly with existing hospital operations.

The physical design in accordance with an embodiment of the invention includes a lightweight, spill-proof cup with a capacity of at least 750mL. This larger capacity in accordance with an embodiment of the invention is carefully chosen to reduce refilling frequency while maintaining manageable weight for patients with weakened grip strength or mobility issues. The cup features double handles to facilitate easy grasping and manipulation by patients with various physical limitations. The invention in accordance with an embodiment of the invention incorporates an improved twist-on lid design that provides easier access through rotational motion rather than vertical lifting, which is particularly beneficial for patients with weakened grip strength, limited dexterity, or mobility issues. To maintain hygiene standards, the preferred embodiment of the invention incorporates either a sterilizable shell or a disposable liner system, allowing for efficient cleaning or replacement between patient uses.

The twist-on lid design in accordance with an embodiment of the invention represents a significant improvement over conventional hospital cup lids that require vertical pulling motion. The twist-on screw top configuration in accordance with an embodiment of the invention utilizes rotational motion for access, which requires less force and provides better mechanical advantage for patients with compromised hand strength. This design feature in accordance with an embodiment of the invention is particularly beneficial for patient populations commonly requiring fluid monitoring, such as those recovering from stroke, experiencing muscle weakness from prolonged hospitalization, or suffering from conditions that affect grip strength and manual dexterity.

At the core of an embodiment of the invention is a small computing device with an integrated screen, which forms the intelligent component of the system. This device is equipped with a fluid flow monitoring mechanism, potentially utilizing flow meter technology, to accurately measure and record the volume of fluid consumed. The computing device is designed to establish a communicative link with the patient's electronic medical records, enabling real-time updates and seamless integration with existing hospital information systems.

The display screen of the preferred embodiment of the invention serves as the primary interface for patients, healthcare providers, and visitors. It presents critical information such as the remaining fluid allowance for the day, time left until the intake limit resets, and, when necessary, a clear indication of NPO (Nil Per Os) status. This visual communication is crucial for ensuring that all parties involved in patient care are aware of the current fluid intake restrictions and progress.

The physical cup design of an embodiment of the invention incorporates several key features to address the unique needs of patients in healthcare settings. The cup is designed with spill-proof features similar to those found in toddler cups, which helps prevent accidental spills and ensures accurate fluid intake monitoring. This feature is particularly beneficial for patients with weakened grip strength or limited mobility, such as those recovering from strokes or other debilitating conditions.

The preferred embodiment of the invention utilizes a lightweight construction to accommodate patients with reduced strength. A notable design element is the inclusion of double handles, which provides enhanced stability and ease of use for weak patients. This thoughtful design consideration allows patients to maintain a secure grip on the cup, reducing the risk of drops and spills while promoting independence in fluid consumption.

An embodiment of the invention has a capacity of at least 750mL, carefully chosen to strike a balance between adequate fluid containment and manageable weight for patients. This larger capacity in accordance with an embodiment of the invention reduces refilling frequency while remaining light enough for weakened patients to handle comfortably, thereby minimizing opportunities for measurement error and workflow disruption that occur with frequent refilling cycles. The 750mL size in accordance with an embodiment of the invention also aligns well with extended fluid intake monitoring requirements in healthcare settings.

To enhance hygiene and facilitate easy cleaning, the preferred embodiment of the invention comprises a disposable straw with a cap. This component can be easily replaced between patient uses, minimizing the risk of cross-contamination. Additionally, an embodiment of the invention incorporates either a sterilizable shell or a disposable liner system. The sterilizable shell option allows for thorough cleaning and disinfection in hospital sterilization departments, while the disposable liner provides a convenient alternative that can be quickly replaced between patients.

The hygiene protocols in accordance with an embodiment of the invention extend beyond basic cleaning to encompass hospital-grade infection control measures essential for multi-patient clinical environments. The sterilization capabilities in accordance with an embodiment of the invention allow for processing through hospital sterilization departments using standard protocols, while disposable liner systems provide an alternative that ensures complete contamination prevention between patient uses. These hygiene features in accordance with an embodiment of the invention distinguish the clinical application from consumer wellness devices that lack the robust contamination control measures required in healthcare settings.

For seamless integration with hospital information systems, an embodiment of the invention further comprises a digital identification graphic such as a barcode or QR code, optionally placed upon on the bottom of the cup. This digital identification graphic can be scanned to link the cup to the patient's electronic medical records and charting systems, enabling accurate tracking and recording of fluid intake data. This feature streamlines the process of associating fluid consumption metrics with the correct patient file, reducing administrative burden and potential errors.

The electronic components of an embodiment of the invention form the core of its intelligent fluid monitoring capabilities. The preferred embodiment of the invention incorporates a small computing device with an integrated screen, serving as the central processing unit and user interface for the system. This compact device in an embodiment is designed to fit seamlessly within an aspect of the cup's structure, providing real-time data processing and display functionality without compromising the cup's ergonomics or usability.

A key feature of an embodiment of the invention is its fluid flow monitoring mechanism. This component is potentially implemented as a flow meter, capable of accurately measuring the volume of fluid consumed by the patient. The flow meter technology enables precise tracking of fluid intake, ensuring that healthcare providers have access to reliable and up-to-date information about the patient's hydration status. This level of accuracy is crucial for patients with conditions such as congestive heart failure, where strict fluid management is essential for effective treatment.

The fluid monitoring mechanism in accordance with an embodiment of the invention preferably utilizes capacitive sensor technology for precise fluid level measurement. Capacitive sensing in accordance with an embodiment of the invention provides liquid level detection by measuring changes in dielectric constant of the cup contents, offering substantially greater accuracy than estimation-based methods or external measurement devices. The capacitive sensor approach in accordance with an embodiment of the invention eliminates the susceptibility to environmental interference and proximity effects that compromise the accuracy of conventional liquid level detection systems.

The capacitive sensor technology in accordance with an embodiment of the invention provides liquid level sensing with resolution capabilities of less than 1mm, representing a substantial improvement over traditional measurement approaches. The sensitivity of the capacitive sensor configuration in accordance with an embodiment of the invention is capable of detecting changes in fluid level of less than 0.5mm, which provides approximately three times the sensitivity required for clinical applications where exact fluid volumes are essential for patient safety and treatment efficacy. This high-precision measurement capability in accordance with an embodiment of the invention eliminates the inaccuracies associated with manual estimation and external measurement devices that have plagued existing fluid monitoring approaches.

The capacitive sensor implementation in accordance with an embodiment of the invention comprises a flexible sensor design that can be applied to the exterior of the cup with self-adhesive backing. The sensor configuration in accordance with an embodiment of the invention connects via a connector to the circuit board when the cup is assembled, facilitating both manufacturing efficiency and maintenance accessibility. This approach in accordance with an embodiment of the invention allows the sensor to be positioned optimally for fluid level detection while maintaining the integrity of the cup's hygienic surfaces.

The level of the fluid in accordance with an embodiment of the invention is measured using a sensor capable of measuring changes in the level of less than 0.5mm. This sensor in accordance with an embodiment of the invention may be a capacitive sensor that detects changes in the dielectric constant of the contents of the cup, an ultrasonic sensor or laser sensor that measures the time it takes for a signal to reflect from the surface of the fluid, a pressure sensor, or other sensors meant for measuring fluid levels. The selection of sensor technology in accordance with an embodiment of the invention focuses the patent protection on the unique clinical application of automated patient fluid intake measurement rather than the specific measurement method employed.

The fluid flow monitoring mechanism in an embodiment of the invention can be implemented using various types of flow meters, each suited to the unique requirements of the cup design and fluid monitoring needs. One potential implementation is the use of a turbine flow meter, which can be integrated into the base of the cup or within the straw mechanism.

In the turbine flow meter configuration, a small turbine is placed in the fluid path. As liquid flows through, it causes the turbine to rotate. The rotation speed is directly proportional to the flow rate, allowing for accurate measurement of fluid volume. This type of flow meter is particularly suitable for the preferred embodiment of the invention due to its compact size and ability to measure low flow rates typically associated with drinking.

Another possible implementation is an ultrasonic flow meter. This non-invasive option uses ultrasonic waves to measure fluid flow without direct contact with the liquid. In this configuration, ultrasonic transducers could be embedded in the walls of the cup, sending and receiving sound waves through the fluid. The time difference between the upstream and downstream ultrasonic pulses is used to calculate the flow rate. This method is advantageous as it has no moving parts, reducing the risk of mechanical failure and making it easier to maintain hygiene standards.

For more precise measurements, especially in medical settings where accuracy is crucial, a Coriolis flow meter could be considered. Although typically larger and more complex, miniaturized versions could potentially be integrated into the base of the cup. Coriolis meters measure mass flow directly, providing highly accurate readings regardless of fluid viscosity or temperature.

Regardless of the specific flow meter type chosen, the integration into the cup structure is designed to be seamless and non-intrusive. For example, in the case of a turbine flow meter, it could be housed in a sealed compartment at the base of the cup, with only the necessary inlet and outlet ports exposed to the fluid path. This design ensures that the flow meter components do not come into direct contact with the consumable liquid, maintaining hygiene and facilitating easy cleaning or sterilization of the cup.

The data collected by the flow meter is transferred to other aspects of the invention through a series of steps. First, the raw data from the flow meter (e.g., rotation speed for a turbine meter or time differences for an ultrasonic meter) is converted into a digital signal by an analog-to-digital converter (ADC) integrated into the cup's electronic components.

This digital signal is then processed by the small computing device within the cup. The device applies calibration factors and algorithms to convert the raw data into meaningful fluid volume measurements. These measurements are then used to update the display in real-time, showing the patient and healthcare providers the current fluid intake status.

Simultaneously, the processed data is prepared for transmission to the patient's electronic medical records (EMR) system. The preferred embodiment of the invention formats this data according to healthcare interoperability standards such as HL7 or FHIR. The formatted data is then transmitted via the cup's wireless or wired connectivity options to the hospital's EMR system, ensuring that the patient's fluid intake information is always up-to-date and accessible to the healthcare team.

This comprehensive approach to fluid flow monitoring and data integration enables the preferred embodiment of the invention to provide accurate, real-time fluid intake tracking. By leveraging advanced flow meter technology and seamless data transfer mechanisms, the invention addresses the critical need for precise fluid management in healthcare settings, particularly for patients with conditions like congestive heart failure where strict fluid control is essential for effective treatment.

The preferred embodiment of the invention establishes a communicative link to the patient's electronic medical records (EMR). This connectivity allows for seamless integration with existing hospital information systems, such as Epic, Cerner, or Meditech. The communicative link enables real-time updates of fluid intake data directly to the patient's charts, eliminating the need for manual data entry and reducing the risk of transcription errors. This feature enhances the efficiency of healthcare delivery by providing immediate access to critical fluid intake information for all authorized healthcare providers involved in the patient's care.

The electronic components of an embodiment of the invention work in concert to create a comprehensive fluid monitoring system. The small computing device processes data from the fluid flow monitoring mechanism, displays relevant information on the integrated screen, and transmits updates to the patient's EMR through the established communicative link. This integrated approach ensures that fluid intake is accurately measured, clearly communicated, and properly documented, addressing many of the challenges associated with traditional fluid monitoring methods in healthcare settings.

The display features of an embodiment of the invention are designed to provide clear, accessible information to patients, healthcare providers, and visitors. The preferred embodiment of the invention incorporates a screen that serves as the primary visual interface for conveying critical fluid intake data. The screen of an embodiment of the invention prominently displays the remaining fluid available for the day. This feature allows patients and caregivers to quickly assess how much more fluid can be consumed within the prescribed limits, promoting adherence to fluid restrictions and supporting effective patient care.

Another key display element, optionally displayed upon the screen in the preferred embodiment of the invention is the time remaining before the fluid intake limit resets. This information helps patients and healthcare providers manage fluid consumption throughout the day, ensuring that intake is appropriately distributed and aligned with medical requirements.

To enhance accessibility and user experience, the preferred embodiment of the invention offers an optional voice feature. This feature can announce the time left for fluid consumption and the amount of fluid remaining within the prescribed limit. This audio capability is particularly beneficial for patients with visual impairments or those who may have difficulty reading the screen.

The display features of an embodiment of the invention work in conjunction with the electronic components to provide real-time, accurate information about fluid intake. By presenting this data in a clear, easily understandable format, the invention supports better fluid management, enhances patient autonomy, and assists healthcare providers in delivering more effective care.

Aspects of the invention in various embodiments comprise specially configured software that facilitates among other attributes the settings of the device. The software and settings of an embodiment of the invention provide a comprehensive suite of features designed to enhance functionality, security, and user experience. The preferred embodiment of the invention includes a menu for configuration options, allowing healthcare providers to customize the device according to patient needs and hospital protocols.

A key security feature of an embodiment of the invention is the lock button, which prevents unauthorized changes to the device settings. This ensures that patients or visitors cannot accidentally or intentionally alter critical fluid intake parameters, maintaining the integrity of the monitoring system.

The preferred embodiment of the invention further comprises an NPO (Nil Per Os) mode setting, which can be activated when a patient is not allowed to consume anything by mouth. When enabled, this mode clearly displays the NPO status on the screen, serving as a crucial reminder for patients, healthcare providers, and visitors. When activated, this feature clearly communicates that the patient is not allowed to consume anything by mouth, which is crucial for pre-surgical patients or those with specific medical conditions requiring fluid restriction.

An embodiment of the invention includes a patient discharge/reset function, allowing healthcare staff to easily prepare the device for a new patient. This feature resets all settings to default values, ensuring accurate monitoring for each new user.

Recognizing that patients may consume fluids outside of the monitored cup, the preferred embodiment of the invention provides a manual fluid intake recording feature. This allows healthcare providers or patients to input additional fluid consumption, ensuring a comprehensive record of total fluid intake.

To support manual charting when necessary, an embodiment of the invention includes a history feature. This function allows healthcare providers to review past fluid intake data, which can be particularly useful in cases where automatic data transmission to the electronic medical records system may have been interrupted.

The preferred embodiment of the invention incorporates a pairing mechanism with hospital charting systems. This feature enables seamless integration with existing electronic medical record platforms such as Epic, Cerner, or Meditech, facilitating real-time data transfer and reducing the administrative burden on healthcare staff.

To accommodate diverse patient populations, an embodiment of the invention offers language options, including Spanish. This multilingual capability ensures that critical fluid intake information is accessible to a wider range of patients, promoting better understanding and compliance with fluid restrictions.

These software and settings features of the preferred embodiment of the invention work in concert to create a versatile, secure, and user-friendly fluid monitoring system. By providing comprehensive configuration options, ensuring data integrity, and facilitating seamless integration with existing hospital systems, the invention addresses many of the challenges associated with traditional fluid monitoring methods in healthcare settings.

The integration features of an embodiment of the invention are designed to seamlessly connect with existing hospital information systems and provide robust data management capabilities. The preferred embodiment of the invention offers compatibility with widely used hospital charting systems such as Epic, Cerner, and Meditech.

The healthcare integration capabilities in accordance with an embodiment of the invention implement specific technical protocols for direct connectivity with hospital electronic medical record systems. The use of HL7 and FHIR interoperability standards in accordance with an embodiment of the invention ensures compatibility with major hospital charting systems including Epic, Cerner, and Meditech, while barcode scanning workflows provide secure patient-device association. The nurse verification mechanism in accordance with an embodiment of the invention incorporates professional oversight requirements that distinguish clinical data management from consumer wellness tracking applications, ensuring that automated measurements meet the data integrity standards required for legal medical records.

This compatibility ensures that the fluid intake data collected by the device can be efficiently integrated into the patient's electronic medical records (EMR) without requiring significant changes to existing hospital infrastructure.

A key integration feature of an embodiment of the invention is the barcode system for data association. The cup is equipped with a barcode on its bottom, which can be scanned to link the device to a specific patient's records.

When a healthcare provider scans this barcode using a compatible scanner or mobile device, it triggers the opening of the patient's records on the external device. This process establishes a direct linkage between the fluid monitoring cup and the patient's EMR, ensuring that all recorded data is accurately associated with the correct patient.

The preferred embodiment of the invention supports various connectivity mechanisms to facilitate communication with EMR systems. Wireless connectivity options may include Wi-Fi or Bluetooth technologies, allowing for real-time data transmission without the need for physical connections. For environments where wireless connectivity may be restricted, the device can also support wired connections through standard interfaces such as USB or Ethernet.

To ensure interoperability with diverse healthcare IT systems, an embodiment of the invention implements data formatting using industry-standard protocols such as HL7 (Health Level Seven) and FHIR (Fast Healthcare Interoperability Resources). For example, fluid intake data could be formatted as an HL7 v2.x message or a FHIR Observation resource. This standardized approach to data formatting facilitates seamless integration with existing EMR systems and ensures that the fluid intake information can be easily interpreted and utilized by healthcare providers across different platforms.

The preferred embodiment of the invention also includes a backup pairing system for data association in cases where barcode scanning may not be feasible or fails. This backup system could involve manual entry of a unique identifier or a secondary wireless pairing method to ensure that the device can always be correctly associated with the patient's records.

To enhance data accuracy and patient safety, an embodiment of the invention incorporates a nurse verification mechanism for double-checking recorded information.

This feature allows healthcare providers to review and confirm the fluid intake data before it is permanently recorded in the patient's EMR. The verification process may include a prompt on the device screen or the connected EMR system, requiring the nurse to acknowledge and approve the recorded data. This additional layer of human oversight helps to catch any potential errors or discrepancies in the automated data collection process.

These integration features of the preferred embodiment of the invention work together to create a comprehensive and reliable fluid monitoring system that seamlessly integrates with existing hospital infrastructure. By leveraging standardized data formats, multiple connectivity options, and robust verification mechanisms, the invention addresses the challenges of accurate fluid intake tracking and timely information sharing in healthcare settings.

Prior art references such as US 10,433,666 B1 to Jovanov and WO 2022/254169 A1 to Smart Hydration represent the current state of consumer wellness and athletic performance tracking devices that fundamentally fail to address the critical requirements of clinical healthcare environments. These consumer-focused systems are primarily designed for personal hydration encouragement and general wellness tracking rather than the precision medical monitoring required for patient safety and clinical decision-making in hospital settings. Jovanov's smart container system, while incorporating capacitive sensing technology, is explicitly directed toward consumer hydration monitoring with features designed for personal wellness rather than clinical applications. Similarly, Smart Hydration's hub-based system with detachable containers is oriented toward general hydration tracking rather than the stringent requirements of medical fluid management.

The precision and accuracy limitations of consumer devices present fundamental barriers to clinical implementation, as these systems typically lack the clinical-grade measurement capabilities required for medical decision-making where exact fluid volumes are essential for patient safety and treatment efficacy. Consumer devices often utilize estimation-based measurement methods or sensors optimized for general tracking rather than the sub-millimeter precision required for clinical applications where fluid restrictions may be measured in small increments critical for conditions such as congestive heart failure or renal failure. The regulatory compliance gap further distinguishes consumer devices from clinical requirements, as personal wellness products are not subject to the medical device regulations, quality management systems, and clinical validation protocols necessary for hospital deployment and integration into patient care workflows.

Consumer devices typically sync with personal smartphone applications rather than hospital electronic medical record systems, creating a fundamental incompatibility with clinical workflows and preventing integration with patient legal medical records. This consumer-focused connectivity approach isolates fluid intake data in personal wellness applications that are inaccessible to healthcare providers and incompatible with hospital information technology infrastructure, requiring manual transcription that reintroduces the very errors and inefficiencies that automated monitoring systems are designed to eliminate. The smartphone-dependent architecture also creates privacy and security concerns in hospital environments where patient data must comply with HIPAA regulations and healthcare interoperability standards that consumer applications are not designed to address.

The motivational and convenience features characteristic of consumer devices, while appropriate for personal wellness applications, are inadequate for the life-critical requirements of clinical fluid management where patient safety depends on precise monitoring and immediate clinical response capability. Consumer smart hydration devices focus on encouragement and habit formation rather than clinical safety protocols such as NPO (Nil Per Os) status monitoring, nurse verification workflows, and integration with clinical decision-making processes that are essential for hospital patient care. The absence of clinical workflow features such as barcode patient-device association, professional oversight mechanisms, and discharge/reset functionality for multi-patient use further demonstrates the fundamental unsuitability of consumer devices for clinical implementation despite their technological sophistication in consumer applications.

The present inventive solution addresses clinical healthcare requirements through purpose-built design specifically engineered for hospital environments, incorporating the unique combination of clinical-grade measurement precision, healthcare workflow integration, and regulatory compliance necessary for medical device implementation. Unlike consumer wellness devices that prioritize convenience and encouragement, the inventive system focuses on patient safety, clinical accuracy, and seamless integration into existing hospital operations through design elements specifically responsive to the limitations and requirements of healthcare settings. The clinical-focused approach encompasses not only the technical measurement capabilities but also the ergonomic, hygiene, and workflow considerations essential for successful implementation in patient care environments.

Integration with hospital Electronic Medical Record systems using healthcare interoperability standards including HL7 and FHIR protocols provides direct connectivity with major hospital charting systems such as Epic, Cerner, and Meditech, enabling real-time data transmission and automatic documentation in patient legal medical records. This enterprise-grade healthcare integration capability distinguishes the inventive solution from consumer devices by implementing standardized healthcare data formatting and transmission protocols that ensure compatibility with existing hospital information technology infrastructure. The healthcare interoperability implementation eliminates the data isolation problems of consumer wellness applications by providing secure, compliant data transmission that meets the regulatory and operational requirements of clinical environments while maintaining the data integrity standards required for medical decision-making.

Clinical workflow features including nurse verification mechanisms and barcode patient-device association provide the professional oversight and patient safety protocols essential for clinical implementation. The nurse verification workflow ensures clinical oversight before permanent recording of fluid intake data in patient medical records, providing a critical safety layer that validates automated measurements through professional healthcare personnel assessment. Barcode patient-device association enables secure linking between the monitoring device and specific patient encounters through hospital authentication protocols, eliminating the risk of data misassociation that could compromise patient safety and ensuring accurate documentation in the correct patient legal medical record.

Regulatory compliance for medical device use in multi-patient clinical environments encompasses the comprehensive quality management systems, clinical validation protocols, and medical device design standards necessary for hospital deployment and patient safety assurance. The inventive solution incorporates medical device regulatory compliance pathways including clinical evidence requirements, safety validation, and quality management systems that ensure consistent performance and reliability in clinical environments. This regulatory framework approach contrasts with consumer devices that lack the medical device compliance necessary for clinical use, providing the foundation for integration into hospital equipment inventories and clinical protocols while meeting the safety and efficacy standards required for medical decision-making applications.

### DETAILED DESCRIPTION: DIFFERENTIATION FROM PRIOR ART

### II. Measurement Precision and Accuracy Requirements

### Problem Paragraph - Estimation-Based Methods

Prior art references such as US 9,382,107 B2 to Pacey rely fundamentally on estimation-based measurement techniques using inclinometer and timing methods rather than direct volumetric measurement of fluid consumption. Pacey's approach utilizes an indirect estimation method that multiplies tilt time by a known flow rate for a given opening size (V=F.t), which inherently lacks the precision required for critical clinical care where exact fluid volumes are essential for medical decision-making. This estimation-based methodology introduces substantial measurement uncertainties that compound over time, making it unsuitable for clinical applications where patients with conditions such as congestive heart failure require precise fluid restrictions measured in small increments.

Jovanov's approach in US 10,433,666 B1, while incorporating capacitive sensing technology, fails to achieve the clinical-grade precision required for medical decision-making applications. Although Jovanov teaches capacitive measurement techniques, the system is explicitly designed for consumer wellness tracking rather than clinical applications, lacking the sub-millimeter resolution capabilities essential for detecting small volume changes critical in clinical care. The consumer-focused design parameters prioritize general hydration encouragement over the exacting measurement standards required for medical fluid management, resulting in insufficient precision for clinical implementation where measurement accuracy directly impacts patient safety and treatment efficacy.

Estimation methods employed in consumer devices are inherently prone to environmental interference and user error, making them fundamentally unsuitable for critical fluid management applications where measurement accuracy is paramount for patient safety. Environmental factors such as ambient vibration, proximity effects from users or external objects, and temperature variations can cause measurement drift and compromise accuracy in estimation-based systems. Consumer-grade sensors lack the sophisticated error correction and environmental compensation algorithms necessary for reliable operation in clinical settings, where measurement integrity must be maintained across varying environmental conditions typical of hospital environments.

Consumer-grade sensors employed in prior art devices fundamentally lack the resolution required for detecting small volume changes essential in clinical care, particularly for patient populations requiring strict fluid management such as those with end-stage renal disease or congestive heart failure. The measurement resolution limitations of consumer devices, typically designed for general hydration tracking rather than medical precision, prevent detection of consumption volumes smaller than 50mL, whereas clinical applications require detection of consumption volumes as small as 5mL for accurate fluid balance management. This resolution gap represents a fundamental barrier to clinical implementation, as healthcare providers require measurement capabilities that exceed the precision standards of consumer wellness devices by an order of magnitude.

The inventive solution utilizes advanced capacitive sensor technology capable of detecting fluid level changes of less than 0.5mm, providing the clinical-grade measurement precision essential for medical fluid management applications. The capacitive sensing implementation employs differential measurement techniques comparing the dielectric constant of fluid-filled versus empty container states, enabling direct volumetric measurement rather than estimation-based approximations. This direct measurement approach eliminates the cumulative errors inherent in estimation methodologies while providing real-time measurement capabilities essential for clinical monitoring applications where immediate feedback is critical for patient safety.

The direct measurement approach implemented in the inventive solution provides approximately three times the sensitivity required for clinical accuracy standards, substantially exceeding the measurement precision capabilities of consumer-grade estimation systems. The capacitive sensor configuration utilizes a segmented array pattern enabling both volumetric measurement and tilt compensation through multi-point differential analysis, providing measurement stability independent of container orientation. This enhanced sensitivity enables reliable detection of small volume changes essential for clinical applications while maintaining measurement accuracy across the varying positional configurations typical of patient use in healthcare environments.

The capacitive sensing configuration eliminates susceptibility to environmental interference through sophisticated differential measurement techniques and temperature compensation algorithms that ensure measurement stability across hospital environmental conditions. The sensor implementation operates at optimized frequency ranges of 100kHz-1MHz to maximize signal penetration through various fluid types while minimizing electromagnetic interference from hospital equipment. Environmental compensation includes temperature adjustment algorithms functional across 15-40° C operational ranges and humidity independence across 20-80% relative humidity environments, ensuring consistent measurement performance in typical hospital settings regardless of environmental variations.

Sub-millimeter precision capabilities of the inventive solution enable detection of consumption volumes as small as 5mL, meeting and exceeding the clinical accuracy requirements for critical fluid management protocols. This measurement precision supports clinical applications requiring strict fluid balance monitoring, including CHF patients with daily fluid restrictions measured in small increments and pre-operative NPO protocols where any fluid consumption must be detected and documented. The clinical-grade precision enables healthcare providers to implement fluid restriction protocols with confidence in measurement accuracy, supporting medical decision-making processes that depend on exact volumetric data for optimal patient outcomes.

Prior art devices fundamentally fail to address the physical limitations of hospitalized patients with weakened grip strength, mobility restrictions, or compromised dexterity that characterize many patient populations requiring fluid monitoring. Standard hospital cups and consumer smart hydration devices lack the ergonomic design features necessary for patients recovering from stroke, arthritis, or general debility associated with prolonged hospitalization, creating barriers to effective fluid intake monitoring. The ergonomic deficiencies of existing devices result in patient difficulty accessing fluids, leading to incomplete compliance with fluid management protocols and compromising the accuracy of consumption tracking essential for clinical care.

Consumer smart cups such as those disclosed in prior art references lack ergonomic features specifically designed for the unique needs of weakened patients, including appropriate grip surfaces, weight distribution, and accessibility mechanisms. These devices are designed for healthy consumer populations and fail to accommodate the physical limitations common in clinical patient populations, including reduced hand strength, limited range of motion, and coordination difficulties. The absence of patient-centered ergonomic design in consumer devices represents a fundamental barrier to clinical implementation, as devices unusable by the intended patient population cannot provide effective fluid monitoring regardless of their technological sophistication.

Standard hospital cups are often too large and heavy for extended monitoring periods, particularly for weakened patients who may struggle with containers exceeding manageable weight thresholds. The size and weight characteristics of conventional hospital fluid containers frequently lead to spills and measurement inaccuracies that compromise data integrity essential for clinical decision-making. Patient difficulty handling oversized containers results in incomplete fluid consumption, spillage during use, and reluctance to maintain adequate hydration levels due to physical challenges associated with container manipulation.

Conventional lid designs requiring vertical lifting force are fundamentally inaccessible to patients with compromised hand strength, arthritis, or coordination limitations that are common in hospitalized patient populations. Standard hospital cup lids often require vertical lifting forces exceeding 15 pounds, which surpass the grip strength capabilities of many patients with medical conditions affecting hand function. This accessibility barrier prevents effective use of fluid monitoring systems by the very patient populations that would benefit most from precise fluid tracking, creating a fundamental design mismatch between device requirements and patient capabilities.

The inventive solution incorporates double handles affixed to the cup body at optimal positions to provide enhanced stability and grip assistance specifically designed for patients with weakened grip strength, tremors, or limited mobility. The double handle configuration positions grip surfaces at 40-70% of total cup height to optimize ergonomic access for bedridden or seated patients, providing multiple grip options that accommodate varying degrees of patient mobility and hand strength. Each handle features grip diameters between 15-25mm optimized for compromised hand function, enabling patients with arthritis, stroke-related weakness, or general debility to maintain secure container control essential for safe fluid consumption and accurate monitoring.

The twist-on lid mechanism utilizing rotational motion requires less than 5 foot-pounds of torque compared to vertical lifting forces exceeding 15 pounds required by conventional hospital lids. The helical threading design utilizes mechanical advantage principles to reduce the force requirements for container access, enabling patients with compromised dexterity to independently open and close the container without assistance. This rotational access mechanism specifically addresses the accessibility limitations that prevent effective use of conventional hospital containers by patient populations with reduced hand strength or coordination difficulties.

The increased 750mL capacity reduces refilling frequency while maintaining manageable weight characteristics that accommodate the physical limitations of hospitalized patients. This volume optimization enables monitoring for complete 6-8 hour nursing shifts with minimal refilling requirements, reducing workflow interruption and measurement discontinuities that compromise data accuracy in existing manual tracking systems. The capacity specification balances the clinical requirement for extended monitoring periods with the physical capabilities of patient populations requiring fluid management, ensuring device usability across varying degrees of patient mobility and strength.

Spill-proof features prevent accidental fluid discharge during patient handling, ensuring measurement accuracy essential for clinical fluid management while accommodating the coordination limitations common in hospitalized patient populations. The spill-resistant design includes controlled-flow openings and leak-resistant assemblies that maintain container integrity during patient manipulation, preventing the fluid loss that would compromise measurement accuracy. These safety features address the practical reality of patient physical limitations while preserving the measurement precision required for clinical applications, ensuring that ergonomic accommodations do not compromise the technical performance essential for medical decision-making.

Consumer smart hydration devices fundamentally lack the hygiene protocols required for multi-patient clinical environments, creating substantial infection control risks that prevent their implementation in hospital settings. Personal wellness bottles designed for individual consumer use cannot be effectively sterilized between patient uses without damaging electronic components, creating cross-contamination risks incompatible with healthcare infection control standards. The materials and construction methods used in consumer devices prioritize cost and convenience over the stringent hygiene requirements of clinical environments, resulting in devices that cannot meet the infection control protocols essential for patient safety.

Personal wellness bottles and consumer smart cups cannot be effectively sterilized using standard hospital sterilization protocols, creating infection control risks that are unacceptable in clinical environments where device sharing between patients is necessary for operational efficiency. Consumer devices typically utilize materials and electronic component integration that cannot withstand the high temperatures, chemical exposure, or radiation levels associated with hospital sterilization procedures including steam sterilization at 134° C, ethylene oxide treatment, or gamma irradiation. The inability to achieve adequate sterilization between patient uses represents a fundamental barrier to clinical implementation regardless of the technological capabilities of consumer devices.

Prior art devices do not address the stringent cleaning and sterilization requirements of hospital environments, where infection control protocols require complete elimination of pathogenic organisms between patient uses. Consumer devices lack the design features necessary for thorough cleaning of all surfaces that may contact patient fluids, including internal passages, sensor surfaces, and electronic component interfaces. The complex geometries and sealed electronic assemblies typical of consumer smart containers prevent access for adequate cleaning and sterilization, creating reservoirs for microbial contamination that pose significant infection risks in clinical settings.

Cross-contamination risks from shared electronic components in consumer-grade devices create unacceptable infection control hazards in clinical environments where device reuse between patients is necessary for cost-effective operation. Consumer devices integrate electronic components directly into container structures that contact patient fluids, creating contamination pathways that cannot be adequately addressed through conventional cleaning procedures. The inability to separate electronic components from patient-contact surfaces in consumer devices prevents effective decontamination and poses substantial infection risks that are incompatible with hospital patient safety standards.

The inventive solution provides dual hygiene approaches specifically designed for clinical environments: sterilizable shells processable through hospital sterilization departments and disposable liner systems that enable complete contamination elimination between patient uses. The sterilizable shell approach utilizes medical-grade polymers capable of withstanding standard hospital sterilization protocols while preserving electronic functionality, enabling complete decontamination between patient assignments. The disposable liner system provides an alternative approach for high-throughput clinical environments where immediate device availability is required, combining complete contamination elimination with preservation of expensive electronic measurement components for extended service life.

Sterilizable components withstand standard hospital protocols including steam sterilization at 134° C, ethylene oxide sterilization at 55° C, and gamma irradiation sterilization up to 25kGy exposure levels without compromising measurement accuracy or device functionality. The medical-grade materials selection ensures compatibility with existing hospital sterilization infrastructure while maintaining the precision measurement capabilities essential for clinical applications. This sterilization capability enables the device to meet hospital infection control standards while preserving the technical performance required for clinical fluid monitoring, addressing the fundamental compatibility gap between consumer devices and healthcare infection control requirements.

The disposable liner system enables complete contamination elimination between patient uses while preserving electronic components through isolation of patient-contact surfaces from measurement electronics. The liner configuration comprises replaceable inner containers manufactured from medical-grade disposable materials, coupled with disposable straw assemblies and sealing components that provide complete barrier protection for reusable electronic systems. This approach enables immediate device availability for subsequent patients without sterilization delays while ensuring complete elimination of cross-contamination risks through replacement of all patient-contact surfaces.

Design features facilitate thorough cleaning of electronic components without compromising measurement accuracy or device functionality through strategic separation of sterilizable and electronic assemblies. Sealing protocols prevent fluid ingress into electronic assemblies while maintaining sensor access required for accurate capacitive measurement through container wall structures. The separation design enables complete cleaning and sterilization of patient-contact surfaces while protecting sensitive electronic components from damage during decontamination procedures, ensuring long-term device reliability and measurement precision in clinical environments.

Prior art devices like Jovanov integrate with personal smartphone applications rather than hospital information systems, creating fundamental incompatibilities with clinical workflows and preventing integration with patient legal medical records. Consumer device connectivity is designed for personal wellness tracking through proprietary smartphone applications that are inaccessible to healthcare providers and incompatible with hospital information technology infrastructure. This consumer-focused connectivity approach isolates fluid intake data in personal wellness applications that cannot be accessed by clinical staff, requiring manual transcription that reintroduces the very errors and inefficiencies that automated monitoring systems are designed to eliminate.

Consumer devices lack compatibility with enterprise-grade healthcare IT infrastructure, preventing integration with the electronic medical record systems that are essential for clinical documentation and medical decision-making. The proprietary communication protocols and data formats used by consumer devices are not designed to interface with healthcare information systems, creating data silos that prevent clinical utilization of monitoring information. Consumer device architecture prioritizes consumer convenience over healthcare interoperability standards, resulting in systems that cannot communicate with the hospital IT infrastructure essential for clinical implementation.

Data isolation in personal wellness apps prevents integration with patient medical records and clinical workflows, creating documentation gaps that compromise patient care continuity and regulatory compliance. Consumer applications store fluid intake data in formats and locations that are inaccessible to healthcare providers during clinical decision-making processes, requiring manual data transfer that introduces errors and delays. The isolation of monitoring data from clinical documentation systems prevents healthcare providers from accessing comprehensive patient information during treatment decisions, limiting the clinical utility of fluid monitoring regardless of measurement accuracy.

Absence of healthcare data standards compliance creates barriers to clinical adoption by preventing consumer devices from meeting the regulatory and operational requirements of healthcare environments. Consumer devices do not implement healthcare interoperability protocols such as HL7 or FHIR that are required for integration with hospital information systems. The lack of healthcare data standards compliance prevents consumer devices from meeting regulatory requirements for medical device data exchange, creating legal and operational barriers to clinical implementation that cannot be resolved through software modifications alone.

The inventive solution implements direct EMR integration using healthcare interoperability protocols including HL7 v2.x and FHIR standards, enabling seamless communication with hospital electronic medical record systems. The healthcare interoperability implementation formats fluid intake observations as standardized HL7 segments within established message structures such as ADT^A08 and ORU^R01, ensuring compatibility with existing clinical documentation workflows. For FHIR-enabled systems, fluid intake data is structured as FHIR R4 Observation resources with appropriate LOINC coding, providing standardized data exchange that meets healthcare industry requirements for medical device integration.

Compatibility with major hospital systems including Epic, Cerner, and Meditech is achieved through standardized data formatting and transmission protocols that meet enterprise-grade healthcare IT requirements. The system implements HIMSS Analytics Stage 7 certification standards for healthcare information technology integration, ensuring compatibility with hospital EMR platforms across varying institutional configurations. This comprehensive compatibility approach enables healthcare institutions to implement fluid monitoring without requiring modifications to existing clinical information systems, facilitating adoption through alignment with established healthcare IT infrastructure.

Barcode patient-device association enables secure linking to specific patient encounters and medical records through hospital authentication protocols that prevent data misassociation errors. The barcode implementation utilizes GS1-compliant encoding standards with Code 128 or Data Matrix symbology to encode unique device identifiers linked to specific patient encounters. The three-step verification process includes device authentication through encrypted handshake protocols, patient identity confirmation via hospital Active Directory integration, and clinical authorization verification ensuring appropriate nursing personnel oversight for device assignment.

HIPAA-compliant data transmission and storage meet healthcare regulatory requirements through implementation of encryption protocols and access controls designed specifically for protected health information. The data transmission architecture utilizes healthcare-grade security protocols including end-to-end encryption, audit trail maintenance, and role-based access controls that ensure patient privacy while enabling clinical access to monitoring data. Compliance with healthcare regulatory standards enables the device to meet institutional requirements for medical device deployment while protecting patient information according to federal healthcare privacy regulations.

Prior art devices lack specialized features for clinical safety protocols such as NPO (Nil Per Os) management, which represents a critical patient safety requirement for pre-operative and medically restricted patient populations. Consumer devices do not provide NPO mode functionality with visual indicators and automatic alerting capabilities essential for preventing inadvertent fluid consumption during periods when patients are medically restricted from oral intake. The absence of NPO protocol support in consumer devices creates patient safety risks in clinical environments where strict adherence to fluid restrictions is essential for preventing anesthesia complications and medical procedure delays.

Consumer devices do not provide mechanisms for clinical oversight and data verification that are essential for ensuring data integrity before permanent recording in patient legal medical records. The automated data collection capabilities of consumer devices lack the professional verification workflows required in clinical environments where healthcare providers must confirm measurement accuracy before documentation in legal medical records. This absence of clinical oversight mechanisms prevents consumer devices from meeting the documentation standards required for clinical decision-making, where data integrity is essential for patient safety and regulatory compliance.

Absence of patient discharge/reset functionality for device reuse in clinical settings prevents effective device lifecycle management in hospital environments where equipment must be efficiently transitioned between patient assignments. Consumer devices designed for individual ownership lack the configuration management features necessary for multi-patient clinical environments, including patient-specific data clearing, system reconfiguration, and device preparation for subsequent assignments. This limitation prevents effective utilization of monitoring devices in clinical settings where operational efficiency requires rapid device turnover between patients.

Missing integration with nursing workflow and clinical decision-making processes prevents consumer devices from supporting the complex care coordination requirements of hospital environments. Consumer devices lack the clinical software features necessary for integration with nursing documentation systems, physician order entry, and clinical decision support tools that are essential components of modern healthcare delivery. The absence of clinical workflow integration limits the utility of monitoring data for healthcare decision-making, preventing consumer devices from supporting the comprehensive care coordination that characterizes effective clinical fluid management.

The inventive solution includes dedicated NPO mode with prominent visual indicators and automated alerting to nursing stations, providing critical patient safety features essential for pre-operative and medically restricted patient populations. When activated, NPO mode implements a multi-layer safety system including prominent visual display indicators, audio alert capabilities for visually impaired patients, and automatic data transmission to nursing station monitoring systems alerting staff to any attempted fluid access during restriction periods. This comprehensive NPO functionality addresses critical patient safety requirements while providing healthcare providers with immediate notification of restriction violations that could compromise patient safety during medical procedures.

Nurse verification mechanism ensures clinical oversight before permanent recording in patient legal medical records through authenticated healthcare personnel access and dual-factor verification protocols. This workflow requires authenticated healthcare personnel access through hospital credentialing systems, implementation of dual-factor verification for high-risk patient populations, and comprehensive audit trail maintenance for regulatory compliance and quality assurance protocols. The nurse verification system provides essential clinical oversight ensuring data integrity standards required for legal medical documentation while maintaining measurement automation benefits that reduce nursing workload.

Patient discharge/reset functionality streamlines device preparation between patient assignments through automated system reconfiguration and comprehensive data management protocols. This feature clears all patient-specific data, resets measurement baselines, performs system calibration verification, and prepares the device for subsequent patient assignment while maintaining comprehensive usage logs for equipment maintenance and infection control tracking. The discharge functionality enables efficient clinical workflow transitions while ensuring data security and device readiness for immediate redeployment in high-throughput clinical environments.

Clinical software features including configuration lock, manual fluid recording, and comprehensive audit trail maintenance provide the workflow integration necessary for effective clinical implementation. The configuration lock mechanism prevents unauthorized parameter modifications through hierarchical access control, ensuring that fluid restriction parameters established by physician orders cannot be altered by patients or visitors without appropriate clinical authorization. Manual fluid intake recording enables healthcare personnel to input additional consumption data from other containers, ensuring comprehensive fluid intake documentation while maintaining measurement accuracy standards required for clinical decision-making.

Smart Hydration's hub-based system with detachable containers introduces measurement discontinuities that compromise the precision required for clinical applications where continuous monitoring is essential for patient safety. The detachable container design creates measurement gaps during disconnection periods that prevent accurate tracking of consumption timing and volume, creating data integrity issues incompatible with clinical requirements. These measurement discontinuities prevent healthcare providers from maintaining the continuous monitoring necessary for conditions requiring strict fluid management, where gaps in data collection can compromise patient safety and treatment efficacy.

Prior art estimation methods cannot achieve the precision required for conditions like CHF where exact fluid volumes determine treatment decisions and medication adjustments. Congestive heart failure patients require fluid restrictions measured in precise increments where variations of even 100mL can trigger acute exacerbations requiring emergency interventions. Estimation-based approaches lack the measurement precision necessary for clinical applications where exact volumetric measurements directly impact medical decision-making, preventing their use in patient populations where fluid management precision is critical for preventing costly medical complications.

Consumer-grade measurement approaches lack the reliability needed for critical fluid management in clinical care, where measurement accuracy directly impacts patient outcomes and treatment decisions. Consumer devices prioritize user convenience over measurement precision, resulting in accuracy standards insufficient for clinical applications where exact fluid volumes determine medication dosing, treatment effectiveness, and patient safety protocols. The reliability limitations of consumer measurement systems prevent their implementation in clinical environments where measurement errors can have significant consequences for patient health and safety.

General hydration tracking systems are optimized for encouragement rather than medical accuracy, creating fundamental incompatibilities with clinical requirements where precise measurement is essential for patient safety. Consumer hydration devices focus on motivation and habit formation rather than the exacting measurement standards required for medical fluid management, resulting in precision specifications inadequate for clinical implementation. The optimization for consumer engagement rather than clinical accuracy creates measurement systems that cannot meet the reliability and precision standards required for healthcare applications where fluid monitoring directly impacts medical decision-making.

The inventive solution achieves resolution capabilities of less than 0.3mm through advanced capacitive sensing technology that provides clinical-grade measurement precision substantially exceeding consumer device capabilities. The capacitive sensor implementation utilizes differential measurement techniques comparing the dielectric constant of fluid-filled versus empty container states, providing measurement resolution approximately three times the sensitivity required for clinical accuracy standards. This enhanced precision enables reliable detection of consumption volumes as small as 5mL, meeting the exacting measurement requirements for critical fluid management protocols where small volume changes have significant clinical implications.

The integrated measurement system eliminates discontinuities from detachable components found in prior art through seamless sensor integration that maintains continuous monitoring throughout patient use. Unlike hub-based systems that create measurement gaps during container disconnection, the inventive solution provides uninterrupted monitoring capability essential for clinical applications requiring continuous data collection. This integrated approach ensures measurement continuity necessary for healthcare providers to maintain accurate fluid balance assessments throughout complete monitoring periods, supporting clinical decision-making processes that depend on comprehensive consumption data.

Temperature compensation algorithms and multi-point differential analysis provide clinical-grade accuracy across hospital environmental conditions through sophisticated measurement adjustment protocols. The sensor configuration operates reliably across 15-40° C operational ranges with humidity independence across 20-80% relative humidity environments typical of hospital settings, ensuring consistent measurement performance regardless of environmental variations. Advanced signal processing algorithms compensate for environmental factors that could affect measurement accuracy, maintaining clinical precision standards across the varying conditions typical of healthcare environments.

Precision measurement capabilities enable detection of 5mL consumption volumes essential for critical fluid management protocols, supporting clinical applications where small volume changes have significant therapeutic implications. This measurement precision supports CHF patients with strict daily fluid limitations, pre-operative NPO protocols where any fluid consumption must be detected, and renal patients requiring precise fluid balance management. The clinical-grade precision enables healthcare providers to implement fluid restriction protocols with confidence in measurement accuracy, supporting medical decision-making processes that depend on exact volumetric data for optimal patient outcomes and prevention of medical complications.

Prior art consumer devices lack medical device regulatory compliance for clinical use, preventing their implementation in healthcare environments where regulatory approval is required for patient care equipment. Consumer wellness products are not subject to the medical device regulations, quality management systems, and clinical validation protocols necessary for hospital deployment and patient safety assurance. The regulatory compliance gap between consumer devices and medical device requirements creates legal barriers to clinical implementation that cannot be resolved through software modifications or workflow adaptations alone.

Absence of quality management systems and clinical validation required for hospital deployment prevents consumer devices from meeting the safety and efficacy standards essential for medical device approval. Consumer devices lack the comprehensive quality management systems including design controls, risk management protocols, and clinical evidence generation that are required for medical device regulatory compliance. The absence of clinical validation data demonstrating safety and efficacy for medical applications prevents consumer devices from obtaining the regulatory approvals necessary for clinical implementation regardless of their technological sophistication.

Consumer devices are not designed to meet FDA medical device requirements or hospital equipment standards, creating fundamental incompatibilities with healthcare institutional requirements. The design and manufacturing processes for consumer devices prioritize cost and convenience over the safety, reliability, and performance standards required for medical device approval. Hospital equipment standards require comprehensive documentation, validation, and quality assurance protocols that are not implemented in consumer device development processes, preventing their acceptance in clinical environments.

Lack of clinical evidence and validation for medical decision-making applications prevents consumer devices from meeting the evidence standards required for healthcare provider acceptance and institutional adoption. Consumer devices lack the clinical studies and validation data demonstrating their safety and efficacy for medical applications, preventing healthcare providers from incorporating them into patient care protocols. The absence of peer-reviewed clinical evidence supporting the use of consumer devices in medical applications creates professional liability and institutional risk management concerns that prevent clinical adoption regardless of device capabilities.

The inventive solution incorporates medical device design standards and regulatory compliance pathways specifically developed for clinical implementation and patient safety assurance. The device development process implements design controls, risk management protocols, and quality management systems that meet medical device regulatory requirements for safety, efficacy, and performance. This comprehensive approach to medical device compliance ensures that the inventive solution meets the regulatory standards necessary for clinical deployment while maintaining the technical performance required for effective fluid monitoring in healthcare environments.

Clinical validation protocols demonstrate safety and efficacy for medical decision-making applications through peer-reviewed studies and evidence generation that meet healthcare standards for device approval. The validation approach includes clinical trials demonstrating measurement accuracy, patient safety, and clinical effectiveness in hospital environments with patient populations requiring fluid management. This evidence-based validation provides healthcare providers and institutions with the clinical data necessary to support adoption decisions and integration into patient care protocols.

Quality management systems ensure consistent performance and reliability in clinical environments through comprehensive design controls and manufacturing protocols that meet medical device standards. The quality management implementation includes design verification and validation, risk management protocols, and manufacturing quality assurance that ensure consistent device performance across clinical applications. These quality systems provide the reliability assurance necessary for clinical implementation where device failure could compromise patient safety and treatment effectiveness.

Compliance with medical device regulations enables integration into hospital equipment inventories and clinical protocols through achievement of the regulatory approvals required for medical device deployment. The regulatory compliance pathway includes FDA medical device approval processes, quality management system certification, and clinical evidence generation that meet healthcare institutional requirements for device adoption. This comprehensive regulatory approach enables healthcare institutions to implement fluid monitoring technology while meeting their obligations for patient safety, regulatory compliance, and risk management in clinical care delivery.

The present inventor has identified several patient populations that would benefit from utilization of the invention in accordance with various embodiments and exemplary intended uses. The intelligent fluid monitoring cup system provides significant benefits for various patient populations requiring strict fluid management, including but not limited to the following:

For congestive heart failure (CHF) patients, the invention in embodiments offers precise tracking of fluid intake, helping prevent fluid overload that can lead to CHF exacerbation. By displaying real-time fluid consumption data and remaining allowances, the system enables patients and healthcare providers to adhere to prescribed fluid restrictions, potentially reducing the risk of hospitalization and associated costs.

The NPO (Nil Per Os) mode feature is particularly beneficial for pre-operative patients. This clear visual indicator helps ensure compliance with pre-surgical fluid restrictions, reducing the risk of complications during anesthesia and surgery.

For patients with end-stage renal failure or those on dialysis, the invention in an embodiment aids in managing fluid intake between treatments. By preventing fluid overload, it can help reduce the strain on dialysis machines and potentially improve treatment efficacy. The system's accurate tracking and display of fluid consumption support patients in maintaining their prescribed fluid limits, potentially mitigating symptoms such as difficulty breathing and swelling.

The clinical integration capabilities of the present invention represent a substantial advancement over consumer wellness devices through implementation of enterprise-grade healthcare information technology protocols. The invention's communicative architecture is specifically designed to integrate seamlessly with major hospital electronic medical record (EMR) systems including Epic, Cerner, Meditech, and other HIMSS Analytics Stage 7 certified platforms.

The healthcare interoperability implementation utilizes industry-standard protocols to ensure secure, compliant data transmission. The HL7(Health Level Seven) integration capability formats fluid intake observations as HL7 v2.x OBX (Observation/Result) segments within ADT^A08 (Update Patient Information) or ORU^R01 (Unsolicited Transmission of Observation Message) message structures. For FHIR-enabled systems, fluid intake data is structured as FHIR R4 Observation resources with LOINC coding 9052-2 (Fluid intake oral estimated) or custom institutional codes for precise volumetric measurements.

The barcode patient association system implements GS1-compliant encoding standards, utilizing either Code 128 or Data Matrix symbology to encode unique device identifiers linked to specific patient encounters. Upon scanning, the system performs a three-step verification process: (1) device authentication through encrypted handshake protocols, (2) patient identity confirmation via hospital Active Directory integration, and (3) clinical authorization verification ensuring appropriate nursing personnel oversight for device assignment.

The capacitive sensing technology represents a significant advancement over estimation-based measurement methods disclosed in prior art consumer devices. The invention's capacitive sensor configuration utilizes differential measurement techniques comparing the dielectric constant of fluid-filled versus empty container states, providing measurement resolution of less than 0.5mm fluid level changes.

The sensor implementation comprises a flexible printed circuit assembly with self-adhesive backing, applied to the exterior surface of the inner cup container. The capacitive sensing elements are arranged in a segmented array pattern, enabling both volumetric measurement and tilt compensation through multi-point differential analysis. This configuration eliminates the susceptibility to environmental interference that compromises conventional liquid level detection systems, providing approximately three times the sensitivity required for clinical accuracy standards.

The sensor's technical specifications include: operating frequency range of 100kHz-1MHz to optimize signal penetration through various fluid types, temperature compensation algorithms functional across 15-40° C operational range, and humidity independence across 20-80% relative humidity environments typical of hospital settings. The measurement precision enables detection of consumption volumes as small as 5mL, substantially exceeding the accuracy requirements for critical fluid management applications.

In an embodiment, the sensor module is configured to distinguish between fluid dispensed from the container for patient consumption and fluid dispensed for non-consumption purposes, such as pouring out unused fluid, rinsing the container, or accidental spillage. The distinction is determined based on one or more of: (a) dispensing rate, wherein a slow, controlled pour characteristic of drinking is distinguished from a rapid pour characteristic of emptying or rinsing; (b) dispensing volume, wherein volumes consistent with a single drink (e.g., 50-250 mL) are classified as consumption events while volumes approximating the full container capacity are classified as non-consumption events; and (c) container orientation, wherein the tilt angle and duration of tilt are analyzed to distinguish a drinking gesture from an emptying or cleaning gesture. When the sensor module classifies a dispensing event as non-consumption, the dispensed volume is excluded from the cumulative fluid intake tracked against the clinician-prescribed fluid intake limit. A non-consumption event is recorded in the event log with a non-consumption flag, enabling clinical staff to review and, if necessary, manually reclassify events through the user interface. This distinction prevents inadvertent inflation of the patient's recorded fluid intake when the container is rinsed, emptied by staff, or otherwise used in a manner not representing actual oral consumption by the patient.

In an embodiment, the system supports tracking cumulative fluid intake across a plurality of container assemblies assigned to the same patient. In a typical clinical scenario, a patient may consume fluid from multiple sources during a single reset period — for example, from a primary water container at bedside, from a smaller cup provided during meals, and from a pitcher used during physical therapy. Each container assembly comprises its own sensor module and processor, and each transmits measured dispensing data to a common patient intake record maintained in the system. The cumulative fluid intake from all container assemblies assigned to the patient is aggregated into a single total and compared against the clinician-prescribed fluid intake limit. When fluid is dispensed from any of the plurality of container assemblies, the aggregated cumulative intake is updated and the remaining fluid allowance displayed on each container's user interface reflects the combined intake across all containers. An alert generated when the cumulative intake approaches or reaches the clinician-prescribed limit is propagated to all container assemblies assigned to the patient, ensuring that the patient and clinical staff are notified regardless of which container triggered the threshold. Container assignment to a patient is managed through a pairing interface accessible to authorized clinical personnel, and containers may be added to or removed from a patient's assignment during the reset period without losing previously recorded intake data.

The ergonomic design features directly address the limitations of standard hospital fluid containers that fail to accommodate patients with compromised physical capabilities. The double handle configuration positions grip surfaces at optimal locations for patients with varying degrees of mobility limitations, including post-stroke weakness, arthritis-related grip reduction, and general debility from prolonged hospitalization.

The twist-on lid mechanism represents a substantial improvement over conventional hospital cup lids requiring vertical lifting force. The helical threading design utilizes mechanical advantage principles, requiring rotational torque of less than 5 foot-pounds compared to vertical lifting forces often exceeding 15 pounds for tightly sealed conventional lids. This design consideration is particularly beneficial for patient populations with compromised hand strength or coordination difficulties.

The 750mL capacity specification addresses the clinical requirement for extended monitoring periods while maintaining manageable weight characteristics. This volume capacity enables monitoring for 6-8 hour nursing shifts with minimal refilling requirements, reducing workflow interruption and measurement discontinuities that compromise data accuracy in existing manual tracking systems.

The hygienic component design addresses the stringent infection control requirements of multi-patient healthcare environments through two distinct approaches: sterilizable shell systems and disposable liner configurations. The sterilizable shell option utilizes medical-grade polymers capable of withstanding standard hospital sterilization protocols including steam sterilization at 134° C, ethylene oxide sterilization at 55° C, and gamma irradiation sterilization up to 25kGy exposure levels.

The disposable liner system provides an alternative approach for high-throughput clinical environments where rapid patient turnover requires immediate device availability. The liner configuration comprises replaceable inner containers manufactured from medical-grade disposable materials, coupled with disposable straw assemblies and sealing components. This system enables complete contamination elimination between patient uses while preserving the electronic measurement and computing components for extended service life.

Both hygiene approaches incorporate design features that facilitate thorough cleaning of electronic components without compromising measurement accuracy or device functionality. Sealing protocols prevent fluid ingress into electronic assemblies while maintaining the sensor access required for accurate capacitive measurement through the container wall structure.

The NPO (Nil Per Os) mode functionality addresses critical patient safety requirements for pre-operative and medically restricted patient populations. When activated, the NPO mode implements a multi-layer safety system including: prominent visual display indicators, audio alert capabilities for visually impaired patients, and automatic data transmission to nursing station monitoring systems alerting staff to any attempted fluid access during restriction periods.

The nurse verification mechanism provides essential clinical oversight ensuring data integrity before permanent recording in patient legal medical records. This workflow requires authenticated healthcare personnel access through hospital credentialing systems, implementation of dual-factor verification for high-risk patient populations, and comprehensive audit trail maintenance for regulatory compliance and quality assurance protocols.

The patient discharge/reset functionality streamlines clinical workflow transitions between patients through automated system reconfiguration. This feature clears all patient-specific data, resets measurement baselines, performs system calibration verification, and prepares the device for subsequent patient assignment while maintaining comprehensive usage logs for equipment maintenance and infection control tracking.

The software architecture implements purpose-built clinical applications that distinguish the invention from consumer wellness tracking devices. The configuration lock mechanism prevents unauthorized parameter modifications through hierarchical access control, ensuring that fluid restriction parameters established by physician orders cannot be altered by patients or visitors without appropriate clinical authorization.

The manual fluid intake recording feature addresses the clinical reality that patients consume fluids from sources beyond the monitored cup system. This functionality enables healthcare personnel to input additional consumption data from other containers, ensuring comprehensive fluid intake documentation while maintaining measurement accuracy standards required for clinical decision-making.

The historical data management system provides clinical decision support through trend analysis, consumption pattern recognition, and predictive alerting for patients approaching fluid restriction limits. Data retention capabilities extend for complete hospital stay durations, enabling comprehensive fluid management assessment and supporting clinical documentation requirements for insurance reimbursement and regulatory compliance.

Burn patients, especially those in the critical first 48 hours post-injury, can benefit significantly from the system's precise fluid monitoring capabilities. The invention in an embodiment allows healthcare providers to input patient-specific fluid allowances based on weight and burn percentage calculations. By ensuring adherence to these carefully determined fluid resuscitation protocols, the system helps prevent both under-hydration and over-hydration, potentially reducing the risk of complications such as pulmonary edema.

The present invention in accordance with an embodiment of the invention differs substantially from consumer smart hydration devices found in the prior art, such as those disclosed in US 2017/0340147 A1 to Leech and US 9,382,107 B2 to Pacey. 3 Consumer devices in accordance with an embodiment of the invention lack essential clinical features including: ergonomic design elements such as double handles for weakened patients; sterilizable shells or disposable liner systems required for hospital hygiene protocols; dedicated NPO (Nil Per Os) mode with clear on-screen clinical indicators; barcode systems specifically designed for linking devices to patient electronic medical records; direct communicative links using healthcare interoperability standards like HL7 or FHIR; and nurse verification workflows essential for clinical data integrity.

The invention in accordance with an embodiment of the invention also distinguishes from indirect clinical monitoring approaches found in the prior art. Clinical hydration monitoring systems like those described in US 2009/0043222 A1 to Licandro attempt to address hydration monitoring through bioelectrical impedance measurement of body segments rather than direct fluid intake measurement from a drinking vessel. 3 This fundamental difference in approach demonstrates that the present invention in accordance with an embodiment of the invention addresses the unmet need for direct oral fluid intake monitoring in hospital settings through a novel combination of precise measurement technology and clinical workflow integration.

In summary, the present invention in accordance with an embodiment provides a comprehensive solution to the longstanding challenges of clinical fluid monitoring through the synergistic integration of advanced sensor technology, thoughtful ergonomic design, and seamless healthcare system integration. The combination of capacitive sensor technology capable of sub-millimeter precision measurement, the improved twist-on lid design for enhanced patient accessibility, and the increased 750mL capacity work together to address both the technical and practical limitations of existing approaches. The invention's clinical workflow features, including NPO mode indication, nurse verification mechanisms, and direct EMR integration using healthcare interoperability standards, transform fluid monitoring from a manual, error-prone process into an automated, accurate, and efficient clinical tool. By incorporating hospital-grade hygiene protocols, ergonomic design elements such as double handles, and comprehensive software functionality, the invention in accordance with an embodiment creates a complete ecosystem that not only measures fluid intake with unprecedented accuracy but also integrates seamlessly into existing hospital workflows. The result is a medical device that has the potential to significantly improve patient outcomes for conditions requiring strict fluid management while reducing healthcare provider burden and minimizing the risk of costly treatment complications associated with inaccurate fluid monitoring. This comprehensive approach to intelligent fluid monitoring represents a substantial advancement over existing solutions and addresses the critical, unmet need for precise, automated fluid intake tracking in clinical healthcare settings.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

I claim:

1. A fluid intake management system for monitoring and restricting fluid consumption of a patient in a clinical setting, comprising:
a container assembly comprising a fluid container and a sensor module configured to measure a volume of fluid dispensed from the fluid container;
a processor operatively coupled to the sensor module;
a non-transitory computer-readable storage medium storing instructions that, when executed by the processor, cause the system to:
receive a clinician-prescribed fluid intake limit for the patient;
track cumulative fluid intake by the patient over a clinician-defined reset period;
compare the cumulative fluid intake against the clinician-prescribed fluid intake limit;
generate an alert when the cumulative fluid intake approaches or reaches the clinician-prescribed fluid intake limit;
implement a lock mode that, when activated by authorized clinical personnel, prevents modification of the fluid intake limit and the reset period by the patient; and
display on a user interface associated with the container assembly at least: the clinician-prescribed fluid intake limit, the cumulative fluid intake, remaining fluid allowance, and time remaining until the next reset period.

2. The system of claim 1, wherein the sensor module comprises at least one of a load cell, a capacitive fluid level sensor, or a flow sensor configured to measure the volume of fluid dispensed from the fluid container.

3. The system of claim 1, wherein the container assembly further comprises a wireless communication module configured to transmit cumulative fluid intake data to at least one of a mobile device application, a clinical monitoring station, or a hospital information system.

4. The system of claim 1, further comprising a nil per os (NPO) mode that, when activated by authorized clinical personnel through the lock mode, disables fluid dispensing tracking and displays an NPO status indication on the user interface, the NPO mode indicating that the patient is restricted from oral fluid intake.

5. The system of claim 1, further comprising an electronic health record (EHR) integration module configured to transmit cumulative fluid intake data to a hospital charting system in a format compatible with the hospital's electronic medical record, enabling automated documentation of the patient's fluid intake in the patient's medical record without manual charting.

6. The system of claim 1, wherein the lock mode comprises a clinician authentication mechanism requiring entry of an authorized credential before the fluid intake limit, the reset period, or an NPO mode setting can be modified, and wherein patient interactions with the user interface are restricted to viewing current fluid intake status information when the lock mode is activated.

7. The system of claim 1, wherein the user interface comprises a display screen integrated into or attached to the container assembly, the display screen configured to present the clinician-prescribed fluid intake limit, the cumulative fluid intake, the remaining fluid allowance, and the time remaining until the next reset period.

8. The system of claim 1, wherein the system is further configured to generate a visual, audible, or haptic alert when the cumulative fluid intake reaches a predetermined threshold percentage of the clinician-prescribed fluid intake limit.

9. The system of claim 1, further comprising a mobile device application configured to receive cumulative fluid intake data from the container assembly and display the cumulative fluid intake, the clinician-prescribed fluid intake limit, and the remaining fluid allowance on a mobile device.

10. The system of claim 1, further comprising a manual intake recording interface accessible through the user interface or a paired mobile device, the manual intake recording interface enabling the patient or clinical staff to record fluid intake from sources other than the container assembly, wherein manually recorded intake is added to the cumulative fluid intake tracked against the clinician-prescribed fluid intake limit.

11. The system of claim 1, wherein the sensor module is configured to distinguish between fluid dispensed for consumption and fluid dispensed for non-consumption purposes based on at least one of dispensing rate, dispensing volume, or container orientation.

12. The system of claim 1, wherein the container assembly is configured as a reusable medical-grade container suitable for hospital use, comprising materials compatible with standard hospital cleaning and sterilization protocols.

13. A method for managing fluid intake of a patient subject to a clinician-prescribed fluid restriction in a clinical setting, comprising:
receiving, at a processor associated with a fluid container, a clinician-prescribed fluid intake limit and a clinician-defined reset period for the patient;
activating a lock mode that prevents the patient from modifying the clinician-prescribed fluid intake limit and the clinician-defined reset period;
measuring, via a sensor module coupled to the fluid container, a volume of fluid dispensed from the fluid container;
tracking cumulative fluid intake by the patient by aggregating measured volumes of fluid dispensed over the clinician-defined reset period;
comparing the cumulative fluid intake against the clinician-prescribed fluid intake limit;
generating an alert when the cumulative fluid intake approaches or reaches the clinician-prescribed fluid intake limit;
displaying on a user interface at least the clinician-prescribed fluid intake limit, the cumulative fluid intake, remaining fluid allowance, and time remaining until the next reset period; and
resetting the cumulative fluid intake to zero upon expiration of the clinician-defined reset period.

14. The method of claim 13, further comprising activating a nil per os (NPO) mode in response to a clinician command, the NPO mode causing the user interface to display an NPO status indication and suppressing fluid allowance information, the NPO mode deactivatable only by authorized clinical personnel.

15. The method of claim 13, further comprising transmitting cumulative fluid intake data wirelessly to at least one of a mobile device, a clinical monitoring station, or a hospital information system.

16. The method of claim 13, wherein the clinician-defined reset period is configurable to align with hospital shift schedules, clinical protocols, or physician-specified intervals, and wherein the cumulative fluid intake resets to zero at the expiration of each reset period without requiring manual intervention.

17. The method of claim 13, further comprising generating a fluid intake history log recording timestamped fluid dispensing events over multiple reset periods, the history log accessible by clinical staff for review and documentation purposes.

18. The method of claim 13, further comprising adjusting the clinician-prescribed fluid intake limit in response to an updated clinical order received through an authorized interface.

19. The method of claim 13, further comprising pairing the fluid container with a hospital charting system such that cumulative fluid intake data is automatically recorded in the patient's electronic medical record.

20. The method of claim 13, further comprising tracking cumulative fluid intake across a plurality of container assemblies assigned to the same patient, such that fluid dispensed from any of the plurality of containers is aggregated into a single cumulative intake total compared against the clinician-prescribed fluid intake limit.

21. A non-transitory computer-readable storage medium storing instructions that, when executed by a processor associated with a fluid container assembly comprising a sensor module, cause the processor to:
receive a clinician-prescribed fluid intake limit and a clinician-defined reset period for a patient in a clinical setting;implement a lock mode preventing patient modification of the fluid intake limit and the reset period;track cumulative fluid intake by aggregating volumes of fluid dispensed as measured by the sensor module over the clinician-defined reset period;generate an alert when the cumulative fluid intake approaches or reaches the clinician-prescribed fluid intake limit;display on a user interface the clinician-prescribed fluid intake limit, the cumulative fluid intake, remaining fluid allowance, and time remaining until the next reset period; andreset the cumulative fluid intake upon expiration of the clinician-defined reset period.

22. The medium of claim 21, wherein the instructions further cause the processor to implement a nil per os (NPO) mode that, when activated, displays an NPO status indication and restricts the user interface to NPO status display only.

23. The medium of claim 21, wherein the instructions further cause the processor to transmit cumulative fluid intake data to a hospital electronic health record system for automated charting.

24. The medium of claim 21, wherein the instructions further cause the processor to generate a visual countdown display showing the remaining fluid allowance decreasing in real time as fluid is dispensed.

25. The medium of claim 21, wherein the instructions further cause the processor to record a manual fluid intake entry received through the user interface and add the manual entry to the cumulative fluid intake.
